(19) United States
(12) Patent Application Publication
(10) Pub. No.: US 2012/0196755 A1
(43) Pub. Date: Aug. 2, 2012

(54) COMPOSITIONS AND METHODS FOR GENOME-WIDE MAPPING OF CHROMOSOME BREAKAGE AND OTHER METHODS FOR MANIPULATION OF CELLS EMBEDDED IN MATRIX

(75) Inventors: Bonita J. Brewer, Seattle, WA (US); Mosur K. Raghuraman, Seattle, WA (US); Wenyi Feng, Seattle, WA (US)

(73) Assignee: UNIVERSITY OF WASHINGTON THROUGH ITS CENTER FOR COMMERCIALIZTION, Seattle, WA (US)

(21) Appl. No.: 13/363,605

(22) Filed: Feb. 1, 2012

Related U.S. Application Data
(60) Provisional application No. 61/438,640, filed on Feb. 1, 2011.

(51) Int. Cl. C40B 20/00; C40B 30/04; C40B 40/00; C12Q 1/68

(52) U.S. Cl. 506/2; 435/6.1; 506/9; 506/13

(57) ABSTRACT

The embodiments described herein provide for compositions and methods for genome-wide mapping of chromosome fragile sites in cellular chromosomal material. More specifically, a method of genome-wide detection of regions of single-stranded DNA and double stranded breaks in DNA, the hallmarks of chromosome fragility, comprises embedding a plurality of cells in a matrix and subsequently directly labeling the single-stranded DNA and/or double stranded DNA breaks; eluting or isolating the labeled chromosomal material from the matrix; and performing analysis to detect the location of the chromosome fragility sites.

### CROSS-REFERENCE TO RELATED APPLICATION

The present specification claims benefit under 35 U.S.C. 119(e) of U.S. Provisional Application No. 61/438,640, filed Feb. 1, 2011, incorporated fully herein by this reference.

### FEDERALLY SPONSORED RESEARCH

This invention was made with U.S. government support under 3K99GM081378, and 3K99GM081378-0251 awarded by the National Institutes of Health. The U.S. Government has certain rights in the invention.

### TECHNICAL FIELD

The present disclosure is directed generally to compositions and methods for genome-wide mapping of chromosome fragile sites and to the manipulation (e.g., direct labeling) of the genetic material of cells embedded in a matrix.

### BACKGROUND

Chromosomal breakage syndromes are relatively rare genetic disorders that are typically transmitted in an autosomal recessive mode of inheritance. In culture, cells from affected individuals exhibit elevated rates of chromosomal breakage or instability, leading to chromosomal rearrangements. The disorders are often characterized by a defect in DNA repair mechanisms or genomic instability, and patients with these disorders show increased predisposition to malignant disorders, particularly lymphoma and leukemia. Additionally, chromosomal breakage can be induced by chemical agents, such as chemotherapies, potentially derailing the course of therapy for cancer victims. Finally, chromosomal breakage may occur following exposure to clastogenic toxins (e.g., asbestos, arsenic, tellurium salts, alkylating agents such as ethylnitrosourea), ionizing radiation (e.g., X-ray, gamma ray, alpha particles), ultraviolet radiation, particular microbes (e.g., measles virus), or other means (e.g., magnetic field, sound waves, cold shock), or combinations of these (e.g., psoralen combined with ultraviolet light).

Chromosome fragile sites (CFS) were first identified in humans as specific regions of constrictions, gaps or breaks on metaphase chromosomes after cells were exposed to chemicals that inhibit DNA replication. Sutherland, 31 Am. J. Human Genet. 136 (1979). As chromosome fragile sites are hot-spots for genomic rearrangement, their identification bears importance on the understanding of mechanisms of genomic instability induced by replication stress. Evidence suggests that delayed or defective replication fork progression through fragile sites may be one of the underlying causes of chromosome fragility. Studies using the model organism, Saccharomyces cerevisiae, have also described chromosome fragility induced by a variety of conditions that either stem from intrinsic properties of DNA templates or from reagents that interfere with DNA replication and/or compromise the checkpoint control mechanism. Cha & Kleckner, 297 Sci. 602 (2002); Lemoine et al., 120 Cell 587 (2005); Admire et al., 20 Genes Devel. 159 (2006); Raveendranathan et al., 25 EMBO J. 3627 (2006); Casper et al., 183 Genetics 423 (2009); Casper et al., 4 PLoS Genet. e1000105 (2008). The unifying hypothesis for observed chromosome fragility is that replication stress causes the destabilization or “collapse” of the replication forks at specific regions of the chromosomes where fragility occurs. The corollary of this hypothesis is that breakage at chromosome fragile sites correlates with altered replication fork progression under the stressed conditions.

Direct and genome-wide evidence that collapsed replication forks give rise to chromosome breakage has historically been lacking, however. Thus, there remains a need for accurate and reliable means for mapping chromosomal breakage sites in suspect genomes in order, for example, to identify those at risk for chromosome breakage disorders and further characterize chromosome breakage at the molecular level throughout the genome.

### SUMMARY

Described herein are methods, compositions and kits for the genome-wide detection and mapping of chromosome fragile sites identified by single-stranded DNA regions (ssDNA) and/or double-stranded breaks (DSB) in eukaryotic cellular chromosomal DNA. The methods described herein permit the direct labeling of single stranded regions and double-stranded breaks in chromosomal DNA in a manner that reflects the presence of these features in the living cell and are indicative of chromosome fragile sites. Briefly, cells embedded in a matrix and treated to render their chromosomal DNA accessible to labeling reagents, are contacted with labeling reagents that directly incorporate label at ssDNA and/or DSBs. Labeled DNA is then isolated from the matrix, and the location and/or degree of breakage or single-strandedness are determined, thereby permitting the detection and mapping of chromosome fragile sites in the cellular chromosomal DNA.

An embodiment of the present invention provides for a method for genome-wide detection of chromosome fragile sites characterized by regions of single-stranded DNA (ssDNA) or double stranded breaks (DSB) in eukaryotic cellular chromosomal DNA, the method comprising the steps of obtaining a matrix in which a plurality of cells have been embedded and said cells' chromosomes have been made accessible to labeling reagents; within the matrix, contacting the chromosomal DNA of the cells with labeling reagents to label directly the DNA at ssDNA regions or at sites of DSB; isolating the labeled DNA from the matrix; and detecting the labeled DNA, wherein the detection of labeled DNA permits identification of the location of the ssDNA region or DSB sites and/or the magnitude of labeling at such sites. The labeling reagents can include random primers for labeling at ssDNA chromosomal regions, and/or comprise Klenow fragment or T4 polymerase for labeling DSB. The labeling reagents can include at least one fluorescently labeled nucleotide, for example, fluorescently labeled dUTP. The labeling reagents can comprise random primers and biotinylated dUTP. In a particular embodiment, the matrix is agarose. The eukaryotic cell can be a yeast cell or a mammalian cell, or more particularly, a human cell. The eukaryotic cell can be a cell of a mammalian cell line, or the cell can be obtained from a mammal suffering from suspected of suffering from a chromosomal breakage disorder. The labeled DNA can, optionally, be fragmented before or after isolating the DNA from the matrix. The labeled DNA can be fragmented by mechanical shearing, enzymatic cleavage, or chemical cleavage. The fragmenting step can be used to generate DNA fragments ranging in size from about 400 bp to about 600 bp, inclusive. Isolating the labeled DNA from the matrix may include electroelution from the matrix or digestion of the matrix. In the instance where the fragmented DNA is biotinylated, the DNA can be isolated by contacting the biotinylated DNA with streptavidin bound to a solid support, whereby labeled DNA fragments are isolated. The detecting step can include contacting the labeled DNA with a microarray, or analyzing labeled DNA using a high throughput DNA sequencing. The detecting step can permit detection of both the location of the chromosome fragile site and the magnitude of labeling at the chromosome fragile site.

Another embodiment provides for a method for mapping chromosome fragile sites in a eukaryotic cell, the method comprising contacting a plurality of eukaryotic cells with an inhibitor of DNA replication; embedding the cells in a matrix; within the matrix, contacting chromosomal DNA of the cells with labeling reagents for a time and under conditions sufficient to permit direct labeling of the DNA at single-stranded regions and/or at sites of double-stranded breaks; isolating labeled DNA from the matrix; and detecting labeled DNA, wherein the detection of labeled DNA indicates the location of single-stranded regions and/or sites of double-stranded breaks in the chromosomal DNA of said cells, whereby one or more chromosome fragile sites are mapped. The method can, optionally, include a step of removing the inhibitor of DNA replication before embedding the cells in the matrix. The labeling reagents can include random primers for labeling at ssDNA chromosomal regions, and/or comprise Klenow fragment or T4 polymerase for labeling DSB. The labeling reagents can include at least one fluorescently labeled nucleotide, for example, fluorescently labeled dUTP. The labeling reagents can comprise random primers and biotinylated dUTP. In a particular embodiment, the matrix is agarose. The eukaryotic cell can be a yeast cell or a mammalian cell, or more particularly, a human cell. The eukaryotic cell can be a cell of a mammalian cell line, or the cell can be obtained from a mammal suffering from suspected of suffering from a chromosomal breakage disorder. The labeled DNA can, optionally, be fragmented before or after isolating the DNA from the matrix. The labeled DNA can be fragmented by mechanical shearing, enzymatic cleavage, or chemical cleavage. The fragmenting step can be used to generate DNA fragments ranging in size from about 400 bp to about 600 bp, inclusive. Isolating the labeled DNA from the matrix may include electroelution from the matrix or digestion of the matrix. In the instance where the fragmented DNA is biotinylated, the DNA can be isolated by contacting the biotinylated DNA with streptavidin bound to a solid support, whereby labeled DNA fragments are isolated. The detecting step can include contacting the labeled DNA with a microarray, or analyzing labeled DNA via high throughput DNA sequencing. The detecting step can permit detection of both the location of the chromosome fragile site and the magnitude of labeling at the chromosome fragile site.

A further embodiment of the present invention provides for a kit for mapping chromosome fragile sites in the chromosomal DNA of a eukaryotic cell, the kit comprising a matrix suitable for embedding eukaryotic cells and reagents for direct labeling of the DNA at single-stranded regions and/or at sites of double-stranded breaks. The kit may also include control cells and/or control DNA. The kit may also include at least one microarray.

### DESCRIPTION OF THE DRAWINGS

FIG. 1A illustrates one embodiment of an outline of procedures for a modified in-gel single stranded DNA (ssDNA) labeling and identification method. FIG. 1B demonstrates that ssDNA persists in mec1 cells recovering from exposure to hydroxyurea (HU). The ssDNA profiles for mec1-1 cells collected after cells were released from a factor arrest and exposed to 200 mM HU for 1 hour (“HU 1 hr”) and after recovering from 1 hour exposure in media without HU for another 1 hour (“R 1 hr”) are shown. The symbols at the top of each graph indicate the locations of replication origins: checked (late/inefficient) origins (squares) and unchecked (early/efficient) origins (circles). See Feng et al., 8 Nat. Cell Biol. 148 (2006). The chromosome numbers are indicated in Roman numerals. Centromere locations are shown as black dots on the X-axis. The same markers are present for all remaining chromosome profiles herein. Color versions of the Figures are available at Feng et al., 1 G3 327 (2011).

FIG. 2A illustrates one embodiment of a methodology of microarray-based genome-wide chromosome breakage mapping. FIG. 2B demonstrates an end-labeled profile of Chr III from a control sample (log phase mec1 cells) that contains in vitro generated BamHI ends. The grey vertical lines indicate known genomic positions of BamHI digestion sites. Arrows indicate potential polymorphic BamHI sites in the yeast strain employed in this study. FIG. 2C demonstrates an in-matrix, end-labeled profile of mec1 cells at “HU 1 hr” (lighter line) and “R 1 hr” (darker line). FIG. 2D demonstrates an end-labeled profile of MEC1 cells at “HU 1 hr” and “R 1 hr.”

FIG. 3 demonstrates a correlation of sites of breakage with ssDNA formation. The chromosome end-labeled profile of sample “R 1 hr” (darker line) resembles the ssDNA profile detected in sample “HU 1 hr” (lighter line). Profiles for Chr II, VI, VII (the left portion), and IX are shown. The positions of Ty elements are indicated by arrows and the category of Ty element is indicated by a label above each peak.

FIG. 4 demonstrates that chromosome breakage is correlated with replication fork progression. FIG. 4A is a schematic representation of the scenarios of replication fork progression and the resulting ssDNA profiles in samples that were released into S phase in the presence of HU at different times. The nomenclature of samples is described in the main text. FIG. 4B illustrates an embodiment of the invention: mec1 cells were released from a factor arrest into medium containing HU at different times as indicated. The cells were exposed to HU for 1 hour followed by recovery in fresh medium without HU for another 1 hour. FIG. 4C represents the CHEF gel electrophoresis of samples from the experimental scheme shown in FIG. 4B. Chromosome breakage is evident in the recovery “R” samples as indicated. The position of Chr IV, which co-migrates with Chr XII, is indicated. FIG. 4D represents Chr IV breakage profiles of recovery “R” samples from cells released into HU at the beginning of the G1/S transition (“T0-R”, lighter line) or after an elapsed 20 minutes (“T20-R”, darker line). FIG. 4E represents the Chr IV breakage profile of the T0-R sample in FIG. 4D overlaid with ssDNA profile of the cells before recovery (“T0-HU”). FIG. 4F represents the Chr IV breakage profile of the T20-R sample in FIG. 4D overlaid with ssDNA profile of the cells before recovery (“T20-HU”). In FIGS. 4D-4F, the left portion of Chr IV is shown in each profile.

FIG. 5 demonstrates that ssDNA predicts sites of eventual chromosome breakage in a mec1-e mutant. MEC1 or mec1-e cells were released from a factor arrest to enter S phase at the restrictive temperature (37° C.), and samples were collected at 40 minutes. Plotted in FIG. 5 are the ssDNA profiles of the MEC1 control sample (lower line) and the mec1-e sample (upper line). The vertical, light grey shaded boxes are replication termination regions that show elevated levels of ssDNA in the me1-e sample.

### DETAILED DESCRIPTION

The following description provides specific details for a thorough understanding of, and enabling description for, embodiments of the disclosure. One skilled in the art will understand that the disclosure may be practiced without these details. In other instances, well-known structures and functions have not been shown or described in detail to avoid unnecessarily obscuring the description of the embodiments of the disclosure. It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” Unless the context clearly requires otherwise, throughout the description and the claims, the words “comprise”, “comprising,” and the like, are to be construed in an inclusive sense as opposed to an exclusive or exhaustive sense; that is to say, in the sense of “including, but not limited to.” Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The embodiments of the invention provides for mapping of sites of chromosome fragility (CFS), e.g., ssDNA and/or DSBs; the use of such mapping to characterize replication fork progression; and the predictive nature of ssDNA on eventual DSB in CFS. In the methods described herein, the chromosomal DNA of cells embedded in a matrix is labeled directly within the matrix at single-stranded sites (ssDNA) and/or at sites of double-stranded breakage (DSB). This approach is applicable to any eukaryotic cell, and particularly to mammalian cells, including human cells. The mapping of ssDNA and DSB can be done simultaneously (i.e., ssDNA and DSB labeling reactions are conducted in the same matrix sample), or in parallel (e.g., one sample is ssDNA-labeled, a separate sample is DSB-labeled, and the two samples compared). In one embodiment, as described herein, replication fork progression was monitored indirectly by detecting ssDNA production at replication forks; and chromosome breakage sites were identified directly by direct mapping of double-stranded breaks (DSBs) using a novel approach. Comparing mapped sites from controls and experimental samples provide valuable information regarding the nature of the chromosome fragility.

The cells in which chromosomal fragility sites (ssDNA regions and/or DSB sites) are mapped may be cells in which such fragility occurs spontaneously (such as cells derived from those suffering from chromosomal breakage disorders or cancers), or the cells may be exposed to agents (e.g., chemicals, radiation), that induce such sites. Example cells include GM06990 lymphoblasts, GM693-cc1 fibroblasts, HEK293T cells, and S. cerevisiae wild-type or mec1-1 cells. Examples of agents that induce chromosomal breakage are replication-impeding drugs like aphidicolin in mammalian cells and hydroxyurea in mammalian and yeast cells. The fragility of the site may be further characterized by removing the inducer and allowing the cells to recover before labeling.

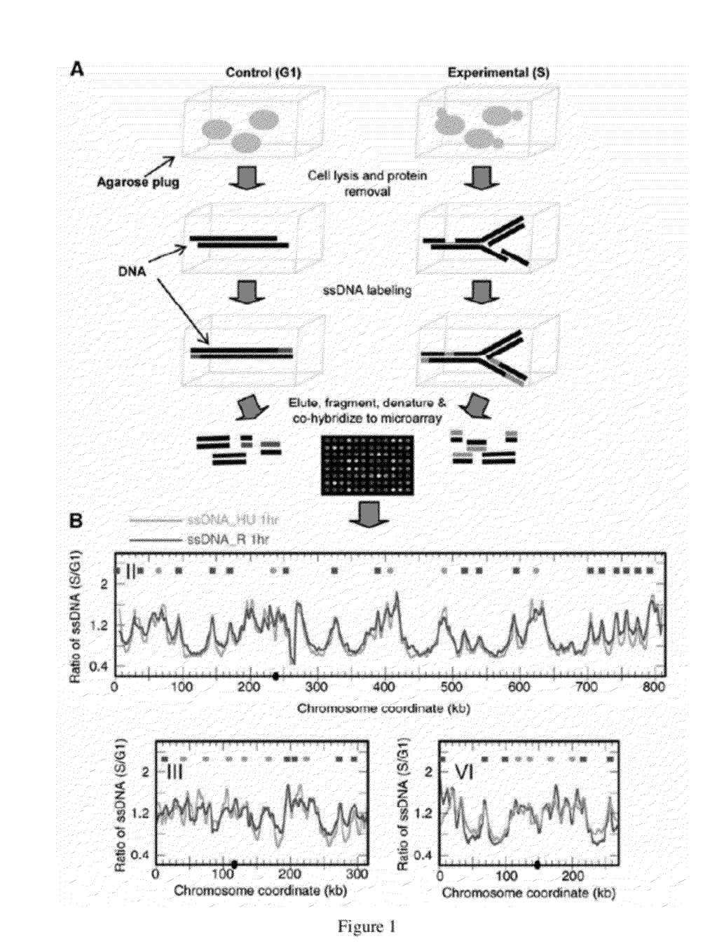

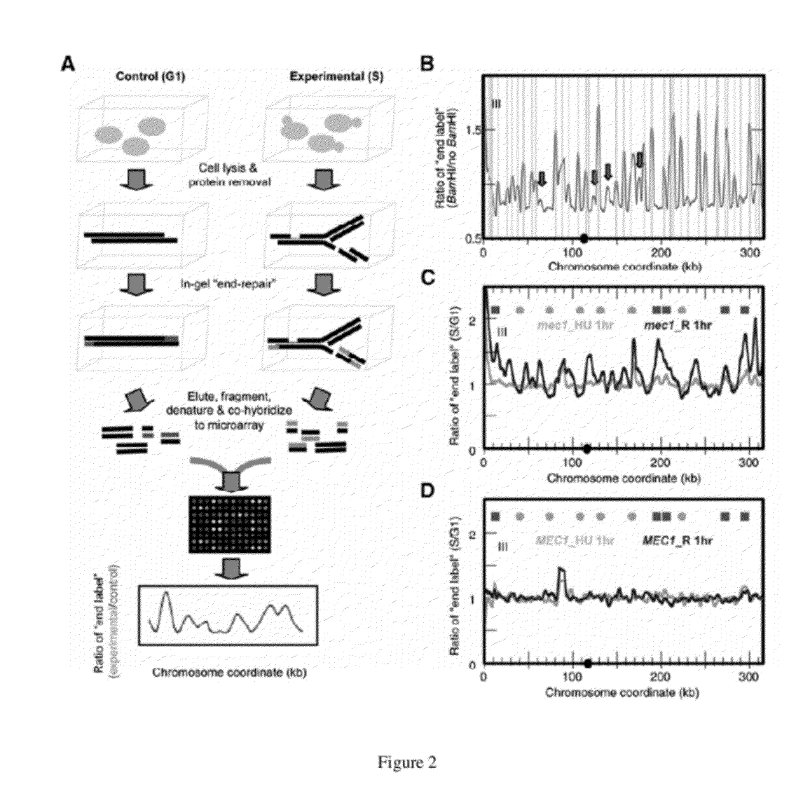

Genomic DNA is prepared by embedding cells in a matrix (e.g., agarose plugs) followed by removal of the cell walls/membranes and proteins (FIG. 1A, FIG. 2A). The matrix of the present invention can be any matrix that stabilizes the cell and cellular milieu sufficiently to inhibit chromosomal damage during DNA preparation and labeling; allows for DNA manipulation such as labeling within the matrix (as described further herein); and allows for the labeled DNA to be obtained from the matrix for further analysis. Agarose, particularly low-melting-point agarose, is an example of such matrix. Other matrices that may be adapted for the present embodiments include nanofibrous or hydrogel scaffolds. Hydrogels can be formed from a vast array of natural and synthetic materials, including collagen, fibrin, hyaluronic acid, composite collagen-chitosan, poly(ethylene glycol), poly(vinyl alcohol), poly(lactide-co-glycolide) and poly(2-hydroxy ethyl methacrylate, and offer a broad spectrum of mechanical and chemical properties. See, e.g., Tibitt & Anseth, 103 Biotechnol. Bioeng. 655 (2009). Labeling of ssDNA and DSBs in the genome in-matrix as described herein minimizes production of DNA degradation and damage that may occur during in vitro manipulation.

The terms “direct labeling”, “directly labeling” or “directly labeled”, as used herein, refer to the result of a genomic DNA labeling method that attaches label to chromosomal DNA while the DNA is embedded in a matrix as described herein. The subject “direct labeling” methods of the present embodiments are not applied to living cells. Rather, direct labeling involves the incubation of a matrix material comprising embedded chromosomal DNA that is or has been made accessible to labeling reagents (i.e., enzyme(s), at least one labeled nucleotide, and optionally additional unlabeled or labeled nucleotides, plus salts, buffers and co-factors as necessary for enzymatic incorporation of labeled nucleotide to the DNA), with such labeling reagents under conditions that permit incorporation of the label into the DNA. Incorporation includes the synthesis of a complement to a single stranded region and/or appending one or more labeled nucleotides or moieties at the site of a double strand break in the DNA. “Direct labeling” as the term is used herein does not include, for example, labeling with dense isotope by culturing the living cells with density labeled nucleotides or nucleotide precursors, although such techniques may be used apart from or in combination with the present embodiments for purposes complementary to the direct labeling of chromosome fragile sites.

The direct labeling of the present embodiments is typically achieved using labeling enzymes. Chromosomal DNA can be labeled within the matrix at single stranded regions by any method known in the art for fill-in DNA synthesis of single stranded regions or templates. Thus, template-dependent polymerase is added to the matrix with one or more, deoxyribonucleotides (dNTPs), of which at least one is labeled, e.g., fluorescently labeled. Where the methods are drawn to the detection of single-stranded regions existing within the chromosome in the cell, it is preferred that the template-dependent polymerase function at, near, or below normal body temperature (e.g., 37° C. for human cells; room temperature or thereabouts for yeast cells, etc.). Thus, while there exists a wide range of thermostable template-dependent DNA polymerases, e.g., Taq polymerase, VENT™ polymerase, etc., it is generally preferred that the polymerase used not require elevated temperature for optimal activity, as elevated reaction temperatures might be expected to artificially expand the size or number of single-stranded regions in the genomic DNA. Non-limiting examples of template-dependent DNA polymerases useful for labeling single-stranded regions include Klenow DNA polymerase (Klenow fragment of E. coli DNA polymerase, which has 5′-3′ polymerase activity and retains 3′-5′ exonuclease (proofreading) activity, but lacks 5′-3′ exonuclease activity), Sequenase™ DNA polymerase (Affymetrix, Inc., Santa Clara, Calif.), and T4 DNA polymerase. An enzyme that has or retains 5′-3′ exonuclease activity (e.g., E. coli DNA polymerase holoenzyme) can be used, but the strand-displacement activity would likely result in broader stretches of labeling than actually occur as single-stranded sequence in the cell. As such, enzymes lacking 5′-3′ exonuclease activity are generally preferred for the methods described herein. Template-dependent synthesis at single stranded regions generally requires a 3′-OH DNA end to extend. This requirement can be supplied by the addition of, for example, random primers; the most frequently used random primers include random hexamers, which are commercially available from a number of sources.

Labeling at double-stranded breaks in the DNA can be achieved with any enzyme or combination of enzymes that use a 5′ or 3′ blunt or recessed DNA end as a substrate for nucleotide addition. Depending on the cause of the break, ends will generally be blunt, 5′ recessed/3′ protruding, or 3′ recessed/5′ protruding. Ideally, a mixture of enzymes or enzyme activities providing, for example, terminal nucleotide exchange, fill-in, or end polishing with exchange activities will be used to label end breaks in a given sample. The ordinarily skilled artisan can select from available enzymes to provide the necessary activities. Ideally, where more than one enzyme is used to provide the necessary activities, the different enzymes will be active under the same reaction and buffer/reactant conditions. Alternatively, enzymes requiring different reaction conditions may be used sequentially. It is also preferred that the enzymes function in the context of the matrix, e.g., in the presence of agarose or low-melting agarose. Activity in the presence of the matrix of choice can be readily determined in a trial reaction with that matrix material and a test template or substrate. Commercially available enzyme mixes effective to label double-stranded DNA breaks include, for example, the enzymes mixes in the END-ITT™ End Repair Kit (Epicentre Biotechnologies, Inc., Madison, Wis.; Cat. No. ER0720; the mixture includes T4 DNA polymerase, as well as T4 polynucleotide kinase), the FAST DNA END REPAIR KIT™ (Fermentas, MD; Cat. No. K0771; the mixture includes T4 DNA polymerase and T4 polynucleotide kinase), and the DNA TERMINATOR™ End Repair Kit (Lucigen, Inc.; Middleton, Wis.).

Any of a number of different types of labels (e.g., radiolabels, fluorescent labels, biotin or other affinity label, etc.) can be used in the methods described herein, the choice generally depending upon the detection platform to be used. For example, where microarray analysis is to be used, fluorescent labels are well suited. Where, instead, a sequencing or whole genome analysis platform is to be used, e.g., an approach suitable to the Illumina/Solexa whole genome sequencing analysis platform, label that permits or facilitates the separation of labeled from non-labeled DNA, such as biotin or another affinity label can be used. In that system, DNA affinity labeled at sites of single-strandedness or at double strand breaks can be contacted with affinity ligand, e.g., streptavidin, on beads or other solid support in order to purify or isolate the labeled DNA from the non-labeled DNA. Non-labeled DNA is washed away and the labeled DNA, once separated or isolated in this manner, can then be processed (adaptors ligated, sequences amplified, etc.) for high throughput sequencing with the Illumina/Solexa or other high throughput sequencing platform. The resulting sequences will identify CFSs or DSBs in the cells tested. Reaction conditions and labeled reagents necessary for the labeling steps described herein are well-known to those of ordinary skill in the art.

As implied by the preceding discussion, direct labeling of chromosomal labeling in-matrix requires that the cells embedded in the matrix have been disrupted and de-proteinized in such a way as to retain stability of the DNA (i.e., minimize artifact DNA damage), and also allow the labeling enzymes and reagents to access (i.e., contact and function at or on) the target ssDNA or DSB to produce the labeled DNA. In other words, that the chromosomal DNA has been made accessible to labeling regents means that the cells have been processed in-matrix such that the DNA and the relevant matrix milieu is compatible with such labeling regents. Approaches to render in-matrix DNA accessible to restriction enzymes are known. Techniques to render in-matrix chromosomal DNA accessible to direct labeling are described herein.

After directly labeling DNA embedded in the matrix as described herein, the labeled DNA is removed from the matrix (e.g, by electroelution, agarase digestion, melting, extraction, etc.), and the labeled DNA subjected to further steps as necessary to identify the genomic locations of the sites of chromosomal fragility (i.e., ssDNA and/or DSB). For example, fluorescently labeled DNA can be directly contacted with, or hybridized to, a microarray comprising fragments representing all or a part of the subject genome. Microarray hybridization methods are well known to those of skill in the art. The resolution of a microarray approach depends upon the size of the subject genome, in that simpler genomes may be fully represented by the features of a single microarray, but more complex genomes may be too large to fit onto a single microarray. For larger genomes, e.g., most mammalian genomes, sub-genomic portions of the genome can be probed on a series of microarrays, e.g., each microarray comprising sequence features drawn from a single chromosome or known portion thereof. Probing a complete set of sub-genomic microarrays with DNA labeled as described herein would provide data regarding all genomic sequences. Microarrays representing sequences from a range of different species are commercially available, e.g., from Affymetrix.

An alternative approach to detection or assignment of the single-stranded or broken sites to their respective genomic locations is to apply high throughput DNA sequencing methods to labeled DNA fragments generated according to the methods described herein. These approaches lend themselves well to affinity-labeled DNA prepared according to the methods described herein that permit the separation of labeled DNA from non-labeled DNA to generate a pool of labeled fragments, each representing a CFS or the site of a DSB. Once the pool of CFS/DSB related fragments is isolated, the DNA can then be subjected to the high throughput DNA sequencing method in order to assign the locations of the fragile or broken sites. Automated Sanger/dideoxy sequencing has been widely used in the past to generate large amounts of sequence data, and can be applied to the material resulting from the labeling methods described herein. Sanger sequencing tends to require greater amounts of starting DNA than newer methods, however, and even in the automated format is slower and more costly on a per nucleotide basis than newer methods. The newer methods are often referred to as “next generation” or “second generation” sequencing, in reference to the Sanger/dideoxynucleotide method as the “first generation” approach. Thus, a number of “next generation” or “second generation” high throughput sequencing techniques have been commercialized, permitting application to the labeled materials prepared as described herein. Available technologies are reviewed, for example, in Metzker, Sequencing technologies—the next generation, 11 Nature Rev. 31 (2010), as well as Shendure & Ji, Next Generation DNA Sequencing, 26 Nature Biotech. 1135 (2008), and Morozova & Marra, Applications of next generation sequencing technologies in functional genomics, 92 Genomics 255 (2008). Various methods provide single molecule sequencing and employ techniques such as pyrosequencing, reversible terminator sequencing, cleavable probe sequencing by ligation, non-cleavable probe sequencing by ligation, and real-time single molecule sequencing. These methods require different types of sample/template preparation, which can include, for example, adapter ligation and amplification, including amplification in droplets in an emulsion, and immobilizing on a solid support (e.g., a bead or slide). The various steps necessary will depend upon the exact platform selected by the user and will be apparent to one of ordinary skill in the art. Devices and systems for performing the second generation sequencing techniques are available from Roche/454 (GS FLX Titanium™), Illumina/Solexa (GA_{II}™), Helicos BioSciences (HeliScope™), Life/APG (SOLiD 3), Pacific Biosciences (PacBio RS™), and Polonator (G.007). In general, long sequence reads should not be absolutely necessary for useful application to the methods described herein, in that any unique sequence can be mapped to its location in the known genome.

The present invention provides for a novel approach to directly map the sites of chromosome fragility (e.g., ssDNA and/or DSB sites) on a genomic level. Previously, a variety of methods for identifying and/or mapping of DSBs generated under a variety of conditions had been reported. None of these methods, however, can do so successfully on a genome-wide scale. For example, indirect end-labeling can be used for fine-scale mapping of a chromosome breakage site once the approximate region of fragility has been identified by genetic experiments. Tandem polymerase chain reaction across a fragile site regions could also identify the breakpoints. Neither method, however, is amenable to CFS mapping on a large scale. On a genome-wide level, meiotic DSBs have been mapped by two microarray-based methods. See, e.g., Buhler et al., 5 PLos Biol. e324 (2007); Cotta-Ramusino et al., 17 Mol. Cell. 153 (2005); Blitzbau et al., 17 Curr. Biol. 2003 (2007); Robine et al., 27 Mol. Cell. Biol. 1868 (2007). Both of these methods, however, have been definitively shown to result in significant amounts of false positives.

Additionally, the “ChIP to chip” method (Chromosome Immunoprecipitation to microarray chip) detects the direct binding of the Spoil protein, and benzoyl-napthoyl-DEAE-cellulose ssDNA enrichment detects the ssDNA tail left by the action of the Spoil protein at the DSBs. Mapping of CFSs can also be achieved indirectly through the detection of phosphorylated γ-H2AX. These methods do not label the terminal DSBs directly, however, and are prone to significant false positives. For instance, there are numerous Spoil binding-sites that are not cleaved. Likewise, with the ssDNA enrichment method, an internal ssDNA gap that is not associated with the DSB will be falsely identified as a product of DSB processing. It has also been reported that γ-H2AX foci were detected in the absence of apparent DSBs measured by Pulse Field Gel Electrophoresis. See Szilard et al., 17 Nat. Str. Mol. Biol. 299 (2010); Banath et al., 64 Cancer Res. 7144 (2004).

The present invention provides for a mapping of the sites of the DSBs on a genomic level, and allows for the correlation of ssDNA and DSBs. In contrast to the prior techniques' demonstrated faults, an embodiment of the invention is a novel chromosome breakage mapping method where DSBs are identified directly (following the scheme outlined in FIG. 2A). To minimize DSBs generated by in vitro manipulations, the novel technique comprises preparing genomic DNA from cells that have been embedded in a matrix (e.g., agarose plugs) and processed for cell disruption (e.g., cell-wall and/or cell-membrane) and protein degradation, and labeling the DSB directly, in-matrix.

The application of the subject mapping methods to a yeast cell line susceptible to HU-induced CFS, the S. cerevisiae mec1 cell/HU model, showed that single-stranded regions of the genome remained single-stranded even after HU removal in mec1 cells (see Example 1). More specifically, the S. cerevisiae mec1-1 checkpoint mutant exhibits chromosome breakage after transient exposure to HU. See Feng et al., 183 Genet. 1249 (2009). When mec1-1 cells are exposed to HU at the beginning of S phase, ssDNA accumulates at the replication forks as a result of origin activation. Then, after the removal of HU, replication forks are incapable of resuming DNA synthesis, and cells generate extensive chromosome breakage. The extensive chromosome breakage provides a model environment in which to test the strengths of the mapping and detection methods described.

Thus, as detailed in Example 1, ssDNA genome maps were prepared by labeling ssDNA in-gel, following the scheme outlined in FIG. 1A. By comparing the in-gel labeled genome maps of “S phase” samples, either HU-exposed cells (HU 1 hr) or HU-exposed cells allowed to recover (R 1 hr), paired with a control “G1 phase” sample, as shown in FIG. 1B, ssDNA was detected near origins of replication in the HU 1 hr sample as observed previously. Interestingly, the ssDNA profile of the R 1 hr sample was virtually identical to that of the HU 1 hr sample, indicating that the single-stranded gaps remained unfilled behind the replication forks even after HU removal.

In the S. cerevisiae mec1-1 model, it is likely that chromosome breakage occurs, at least in part, as a consequence of chromosomes being under persistent tension exerted by the mitotic spindle: breakage was evident near a centromere located on a chromosome capable of bi-orientation on the spindle (CEN2) but not near a centromere on a chromosome unable to achieve bi-orientation (CEN4). Feng et al., 2009. It is unlikely that the chromosome breakage occurred as a direct consequence of the mechanical force exerted by the spindle, but instead occurred as a result of a more open and vulnerable chromatin environment due to spindle extension. The genome mapping via in-gel labeling of DSB in this model showed that chromosome breakage was not restricted to the centromere-proximal regions.

Additionally, if the ssDNAs that persists after removal of HU contributes to chromosome fragility, then their locations should correspond to sites of breakage. Thus DSB genome maps were prepared by labeling DSB in-gel, following the scheme outlined in FIG. 2A. DNAs derived from an equal number of cells from the experimental sample (e.g., R 1 hr sample that contains DSBs) and from a control sample (non-replicating DNA without apparent breaks) were labeled differentially (e.g., with Cy3- and Cy5-conjugated dUTPs) in-gel. The DNA was then eluted from agarose plugs and, and after sonicating the DNA to reduce the average fragment size, co-hybridized to Agilent DNA microarrays. The relative levels of DSBs were expressed as the ratio of fluorescence signals from the experimental sample over those from the control sample.

To validate the in-matrix labeling for genome mapping, as a positive control indicating that the in-gel direct DSB labeling was successful, chromosome breakage was mapped in samples containing DSBs at known sites. Specifically, DNA embedded in agarose plugs was prepared from exponentially growing mec1 cells, and the DNA digested in-gel with the restriction enzyme BamHI. The digested DNA was then labeled in-gel with Cy3-dUTP, and the undigested control DNA was labeled with Cy5-dUTP. Microarray comparison revealed that the “chromosome breakage profile” of the mapped DSBs correlated nearly perfectly with the known BamHI sites in the genome (FIG. 2B). These results also demonstrated that the methods of in-gel preparation and labeling of DNA disclosed herein did not result in extensive site-specific shearing of the DNA: significant breakage elsewhere on the chromosome was not detected.

The specificity of the DSB labeling (via end-repair reaction) was assessed for the DSB DNA ends versus internal stretches of ssDNA that are not associated with DSBs. Previous observations indicated that chromosomes were not breaking in mec1 cells during their exposure to HU, but did contain extensive ssDNA (FIG. 1B). If an internal ssDNA stretch were to contain an adjacent free 3′-OH group, it might be a suitable template for the T4 DNA polymerase. Thus, labeling of any internal stretches of ssDNA by the end-repair reaction would skew the results of breakage mapping. As shown in FIG. 2C, however, the HU 1 hr sample exhibited a rather flat labeling-profile and did not show significant signal at the ssDNA regions near the origins. Thus, the in-gel end-repair reaction does not label internal stretches of ssDNA efficiently.

The present invention also demonstrates that, in the yeast model, chromosome breakage occurs at ssDNA locations. In particular, the novel chromosome breakage mapping method showed that the mec1 sample contained extensive chromosome breakage (R 1 hr), revealing specific labeling at multiple internal sites along chromosomes (FIG. 2C). That the magnitude of the internal labeling was comparable to the labeling at BamHI sites supports the conclusion that these are bona fide sites of in vivo DSB formation. In contrast, the wild-type (WT) cells in both the HU 1 hr or the R 1 hr samples exhibited no significant levels of chromosome breakage as they generated low and flat profiles similar to those seen for mec1 cells in HU for 1 hour.

The present embodiments provide for exquisite characterization of chromosomal breakage at the molecular level. For example, at first glance, the sites of breakage in mec1 cells recovering from HU might appear to occur at specific locations with no obvious correlation to known origins or other landmarks. When parsing origins into the categories of early/efficient (origins that fire in HU in the presence of an activated checkpoint; unchecked origins) and late/inefficient (origins that are delayed in firing after activation of the checkpoint; checked origins), breakage sites are better correlated with the checked origins (FIG. 2C, squares) than with the unchecked origins (FIG. 2C, circles). To perform this comparison, those chromosome positions where significant (above median level) breakage had occurred were identified. Then, the percentage of these chromosome positions within 6 kb of the nearest origin of replication was determined. A 6 kb cut-off was selected, because the Lowess smoothing window size of the microarray data was set at 6 kb.

In the mec1 mutant, all origins fire (Feng et al., 2009), and it appears that the temporal pattern of origin activation remains as replication forks have migrated away from the unchecked (early) origins but are still in the vicinity of the checked (late) origins. This result demonstrates that chromosome breakage correlates with replication fork progression.

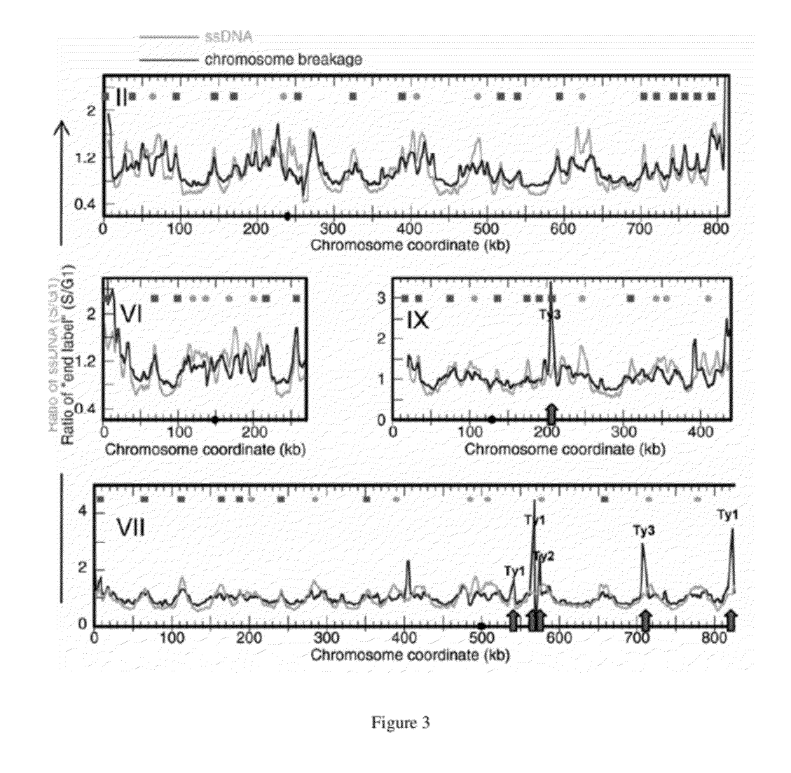

Comparison of the breakage profile of the R 1 hr sample with the ssDNA profile of the HU 1 hr sample (which records the positions of replication forks) reveals striking similarity (FIG. 3). Because the regional accumulation of ssDNA in HU occurs before breaks are detected in the recovery phase, it confirms that ssDNA formation precedes the occurrence of chromosome breakage and dictates their location. Importantly, the identification of ssDNA regions in the genome can be used to predict sites of chromosome breakage, an observation that has profound implication for the study of chromosome fragile sites in the human genome.

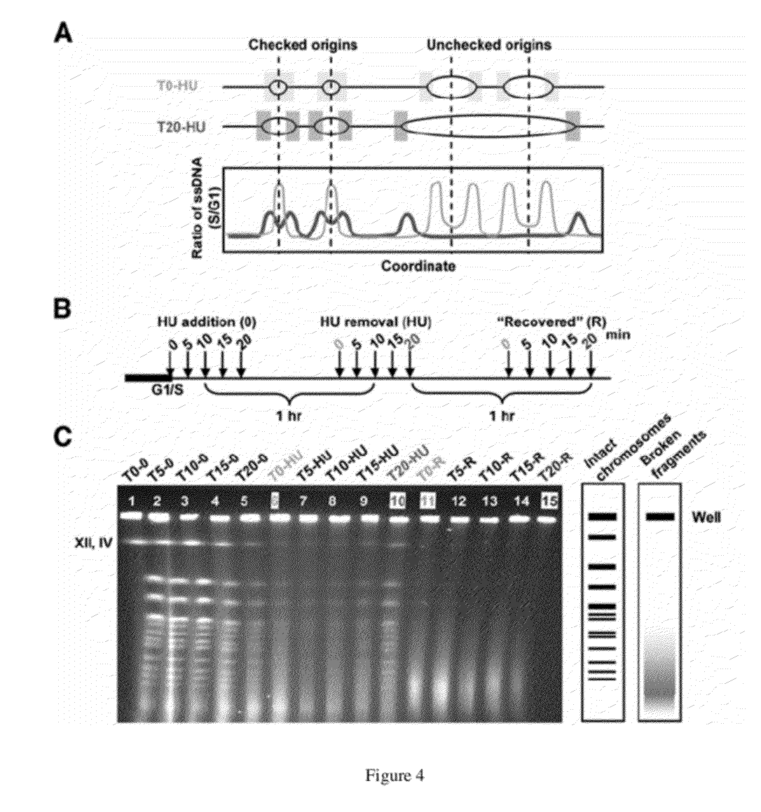

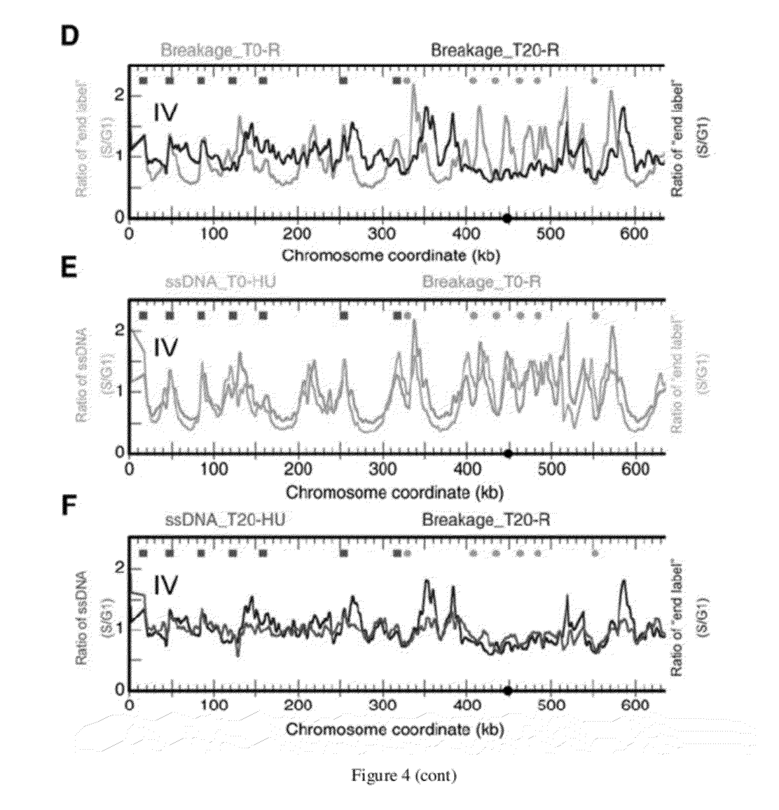

Thus, chromosome breakage is correlated with replication fork progression in this yeast model. As demonstrated herein, the locations of chromosome breakage are correlated with ssDNA production at sites of irreversibly stalled replication forks, providing the first direct evidence that collapsed replication forks lead to DSBs. In another experiment, replication forks were allowed to travel different distances from the origins before HU treatment to generate collapsed forks at varying distances from the origins. The sites of chromosome breakage in samples recovering from HU were then measured (FIG. 4A). HU was added at different times after S phase began, and then the distribution of both ssDNA and double stranded breaks after cells recovered from HU were examined. Because it is the ssDNA at forks that contribute to chromosome fragility, the break sites are then found at a greater distance from these origins. This finding is illustrated in FIG. 4B.

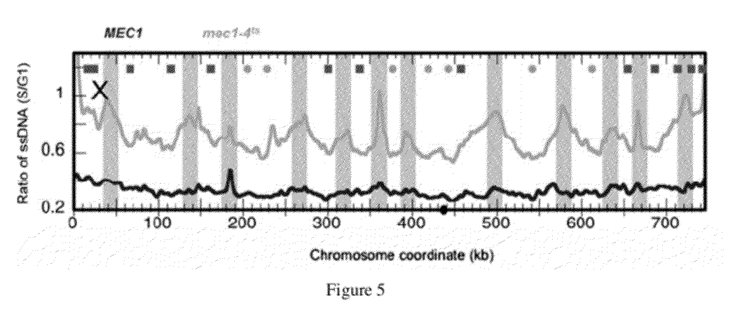

The present invention revealed that ssDNA is detected prior to chromosome breakage in a mec1 temperature-sensitive mutant, without external replication stress. Additionally, ssDNA at other replication stress-induced chromosome breakage sites were detected prior to breakage taking place. As demonstrated herein, ssDNA formation is a common precursor to DSBs as a result of replication stress. It had been reported that a temperature-sensitive mec1-4 allele has defective replication fork progression at the restrictive temperature, without external challenges by HU, and experiences chromosome breakage later in the cell cycle. Cha & Kleckner, 2002. The breakage occurred at regions of the genome described as “replication slow zones” (RSZs), which are primarily replication termination sites. Hence, if and when ssDNA formation could be detected in the RSZs was explored. ssDNA was mapped in the mec1-4 cells as well as the WT control MEC1 cells in mid-S phase (40 minutes after release from the G1 arrest). As shown in FIG. 5, MEC1 cells showed background level of ssDNA, indicating that replication forks were not stalled at specific regions of the genome. In contrast, the mec1-4 cells showed distinct patterns of elevated levels of ssDNA near the replication termini: the very regions where chromosome breakage was shown previously to take place. Cha & Kleckner, 2002.

As disclosed herein, the present invention provides for a genome-wide chromosome breakage mapping method that identifies chromosome breakage using in-matrix DNA labeling of ssDNA and/or DSBs. One embodiment successfully identified chromosome breakage in checkpoint-deficient mec1-1 cells after exposure to and recovery from HU. In the yeast model, chromosome breakage is correlated with replication fork progression, thus providing direct evidence that chromosome fragility results from destabilization of the replication forks in the absence of the checkpoint when the nucleotide pool is reduced/depleted. Additional aspects of the disclosure demonstrated that prior to the occurrence of chromosome breakage, ssDNA formation could be detected at the sites of breakage. Accordingly, as demonstrated, ssDNA formation is a common precursor to chromosome fragility during replication stress. Another embodiment, using a mec1 temperature sensitive allele that exhibits chromosome fragility without external challenge to the replication forks, demonstrated that ssDNA formation was indeed detectable at the sites of eventual chromosome breakage, further confirming that persistence of ssDNA is a precursor to chromosomal breakage. Thus, detection of ssDNA can be used as a tool to identify sites of chromosome fragility, and may signal when particular replication forks encounter impediments and/or become unstable.

The molecular details of some types of ssDNA gaps formed during replication stress remain to be fully characterized. For example, gamma radiation-induced DNA breaks are reported to contain 3′-termini that either contain a phosphoryl group, or a contain group that is neither hydroxyl nor phosphoryl in nature. Henner et al., 257 J. Biol. Chem. 11750 (1982). If the HU-induced single-stranded gaps contain either of these 3′ modifications, they might not be a preferred template for the 3′-to-5′ exonuclease activity of the T4 DNA polymerase. Alternatively, the 3′-OH at the junctions of the internal gaps of ssDNA and the duplex DNA might not be a suitable template for T4 polymerase. It remains to be determined what types of chemical moieties are present at the termini of the ssDNA gaps in the genome. Nevertheless, other polymerases or labeling techniques may be adapted for in-matrix use in such instances. Regardless, the present embodiments are not bound by theory regarding the molecular details of the ssDNA.

Another embodiment of the invention demonstrates the application of the methods to mammalian genomes, such as a human genome. In mammalian cells, ssDNA and DSBs can be induced with agents such as hydroxturea and aphidicolin. The chromosome breaks can be labeled in-matrix, for example, with biotinylated nucleotides. After elution and optional shearing of the DNA, the biotinylated labeled ends are captured on streptavidin beads, followed by ligation to adaptamers, and are then used as template for PCR to amplify the signal. The end readout can be either microarray or high-throughput sequencing. This embodiment is useful, inter alia, for diagnosing chromosome breakage disorders, or for high-throughput screening of suspected clastogenic agents.

Identifying chromosome fragility and mapping such locations within the genome is advantageous in a number of applications, including disease diagnosis and characterization, and screening the potential clastogenic activity of compounds in high-throughput screening.

For example, chromosomal breakage is implicated in several human disorders. More specifically, Ataxia-telangiectasia (A-T) is a syndrome with DNA-repair/processing defects. It is autosomal recessive and results in a complex, multisystem disorder characterized by progressive neurologic impairment, cerebellar ataxia, variable immunodeficiency with susceptibility to sinopulmonary infections, impaired organ maturation, x-ray hypersensitivity, ocular and cutaneous telangiectasia, and a predisposition to malignancy. Bloom syndrome (congenital telangiectatic erythema) is a rare autosomal recessive disorder characterized by telangiectases and photosensitivity, growth deficiency of prenatal onset, variable degrees of immunodeficiency, and increased susceptibility to neoplasms of many sites and types, especially leukemias and lymphomas. Xeroderma pigmentosum is characteristized by dry, pigmented skin, manifested due to a cellular hypersensitivity to ultraviolet radiation resulting from a defect in DNA repair. Nijmegen breakage syndrome is also an autosomal recessive disease in which a mutation in the NBS1 protein causes NBS1 to fail to check the cell cycle in S phase and/or fails to initiate DNA repair; hence NBS individuals suffer radiation sensitivity and a strong predisposition to lymphoid malignancy.

Mutations in NBS1 are also associated with Fanconi anemia, a rare inherited bone marrow failure syndrome. In the early 1960s, several groups observed that cultured cells from patients with Fanconi anemia had increased numbers of chromosome breaks; later, the breakage rate was found to be specifically increased by the addition of DNA cross-linkers, such as diepoxybutane or mitomycin C. This finding led to the identification of patients with Fanconi anemia and aplastic anemia without birth defects, and the diagnosis of Fanconi anemia in patients without aplastic anemia but with abnormal physical findings. Possible complications of Fanconi anemia include hemorrhages, infections, leukemia, myelodysplastic syndrome, liver tumors, and other cancers. Because there is a link between the frequency of chromosomal breaks and the increased propensity for malignancies, chromosomal breakage mapping is a valuable tool in diagnosing and monitoring the progression of these disorders. In particular, the mapping of ssDNA regions and/or DSBs provides for early detection of chromosome fragile site-associated events that may give rise to carcinomas.

Regarding high-throughput screening, normal or other target cells and appropriate control cells are segregated into experimental and control groups, replication phase synchronized, and cells are exposed to the test agent under circumstances sufficient to induce ssDNA regions or DSBs if the agent is, indeed, clastogenic. Optionally, following exposure, possible repair can be allowed by removing the test agent from cell contact. The location of ssDNA and DSBs are then elucidated as described herein, which indicate sites of induced chromosome fragility.

Another aspect of the present embodiments provides for kits useful in mapping CFS. Such kits can include matrix material, labeling reagents, and reference cells or DNAs to provide, for example, negative and/or positive controls. The kit can be in any configuration well known to those skilled in the art and is useful for performing one or more of the methods described herein for the detection and/or quantitation and mapping of at least CFS. The kits are convenient in that they supply many, if not all, of the essential reagents for mapping at least one CFS in a test biological sample (e.g., a cell line, tumor biopsy, serum sample). The kit may contain a microarray. In addition, the assay may be performed simultaneously with a standard or multiple standards included in the kit, such as a predetermined amount of labeled ssDNA or labeled DSB DNA, so that the results of the test can be quantified or validated.

Another embodiment provides a method for monitoring treatment efficacy of a subject with a chromosome fragility disorder, the method comprising: (a) determining, from a biological sample obtained from a subject at a first time point, the location of CFS; (b) determining a location of CFS from a sample obtained from the subject at a second time point; and (c) comparing the location of CFS at the second time point with the location of CFS at the first time point, wherein a decrease in the locations of CFS at said second time point indicates the treatment is efficacious for said subject, and wherein an increase in the locations of CFS at said second time point indicates the treatment is not efficacious for said subject.

Another embodiment provides a method for monitoring treatment efficacy of a subject with a cancer being treated with an agent that has clastogenic side effects, the method comprising: (a) determining, from a biological sample obtained from a subject at a first time point, the location of CFS; (b) determining a location of CFS from a sample obtained from the subject at a second time point; and (c) comparing the location of CFS at the second time point with the location of CFS at the first time point; wherein a decrease in the locations of CFS at said second time point indicates the treatment is not inducing CFS; and wherein an increase in the locations of CFS at said second time point indicates the treatment is inducing CFS for said subject, and alternative treatment strategies may be considered.

The term “subject” in the context of treatment refers to any individual or patient to which the subject methods are performed. Generally the subject is human, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and non-human primates (including monkeys, chimpanzees, orangutans and gorillas) are included within the definition of subject.

The present invention therefore provides for systems (e.g., comprising computer readable media for causing computer systems) to perform methods for mapping CFS in a test genome. Hence, another aspect relates to a computer readable storage medium having computer readable instructions recorded thereon to define software modules for implementing on a computer a method for mapping at least one ssDNA or DSB site of at least one test genome, the computer readable storage medium comprising: (a) instructions for storing and accessing data representing at least one mapped ssDNA or DSB obtained from at least one test genome; (b) instructions for comparing the mapped CFS locations with reference data stored on the storage device using a comparison module, wherein the comparing step produces a retrieved content, and (c) instructions for displaying a page of the retrieved content for the user, wherein the retrieved content displays if there is a change in the locations of the mapped CFS, thereby determining whether the test genome has CFS associated with a chromosomal breakage disorder.

The computer readable storage media can be any available tangible or physical media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), USB memory, a hard disk, flash memory or other memory technology, tablet devices, smartphone devices, CD-ROM (compact disc read only memory), DVDs (digital versatile disks) or other optical storage media, transmission media such as those supporting the Internet or an intranet magnetic cassettes, magnetic tape, magnetic disk storage or other magnetic storage media, other types of volatile and non-volatile memory, and any other tangible medium which can be used to store the desired information and which can accessed by a computer, including any suitable combination of the foregoing. As used herein, computer readable storage media or a computer-readable physical memory does not include, for example, non-tangible, transitory forms of signal transmission, such as radio broadcasts, electrical signals, light pulses, carrier waves, and the like.

Systems and computer readable media described herein are merely illustrative embodiments of the invention for performing methods of, for example, assessing whether a subject has a chromosomal breakage disorder, and are not intended to limit the scope of the invention. The modules of the machine, or those used in the computer readable medium, may assume numerous configurations. For example, function may be provided on a single machine or distributed over multiple machines. Variations of the systems and computer readable media described herein are possible and are intended to fall within the scope of the invention.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples are intended to demonstrate aspects of the disclosure more fully without acting as a limitation upon the scope of the disclosure, as numerous modifications and variations will be apparent to those skilled in the relevant art.

### Example 1

### In-Matrix Labeling to Detect ssDNA and DSBs in Yeast Cell Chromosomes

Yeast strains HM14-3a (MATa RAD53 bar1-1 his6 leu2-3,112 trp1-289) and WFA34 (MATa RAD53::rad53K227A(KanMX4) bar1-1 his6 leu2-3,112 trp1-289) are derivatives of RM14-3a in an A364a background generated through gene conversions as described previously. Feng et al., 2006. BY2006 (MATa mec1-1::HIS3 his3 leu2 trpl ura3) is also an A364a derivative. RCY378 (MATa ho::LYS2 mec1::LEU2 lys2 ura3 leu2::hisG ade2::LK his4x arg4NdeI::mec1-4-kanMX4). Cha & Kleckner, 297 Sic. 602 (2002). The isogenic MEC1 derivative RCY301 was also used. Cells were grown at 30° C. in synthetic complete medium unless otherwise indicated. α factor was used at 200 nM for bar1 strains and 3 μM for BAR1 strains. Pronase was used at 25 μg/ml and 300 μg/ml for bar1 and BAR1 strains, respectively, to remove a factor from the culture medium. HU was added at 200 mM.

Yeast mec1 cells were synchronized at the G1/S boundary using the α factor mating pheromone, followed by release into S phase in the presence of 200 mM HU, and then samples were collected at the beginning (0 hr) and after 1 hour exposure to HU (HU 1 hr). The cell culture was filtered to remove HU, and the collected cells allowed to “recover” in fresh medium without HU, after which samples were collected after 1 hr (R 1 hr). An “S phase” sample, either HU 1 hr or R 1 hr, was paired with a control “G1 phase” sample, and the two samples were differentially labeled with Cy-conjugated dUTPs as described here and over-viewed in FIG. 1A.

Labeling of ssDNA in the genome was conducted in-gel to minimize production of ssDNA that may occur during in vitro manipulation. In-gel ssDNA labeling was achieved as follows. Approximately 5×108 cells were collected for each sample during the time course of HU exposure and subsequent recovery. The cells were then embedded in agarose plugs and spheroplasted using the same procedures as in the CHEF gel electrophoresis analysis. The agarose plugs were pre-equilibrated in 10 mM Tris-HCl, pH 8.0, 0.1 mM EDTA (5 ml per plug) for 30 min, followed by equilibration in 5 ml of 50 mM Tris-HCl pH 6.8, 5 mM MgCl2, and 10 mM β-mercaptoethanol for 30 min at room temperature. An “S phase” sample, either HU 1 hr or R 1 hr, was paired with a control “G1 phase” sample, and the two samples were differentially labeled with Cy-conjugated dUTPs. For each sliver of agarose plug containing 108 cells in approximately 50 μl, 50 μl of labeling mix (50 mM Tris-HCl pH 6.8, 5 mM MgCl2, and 10 mM β-mercaptoethanol, 0.24 mM of each of dATP, dCTP, and dGTP, 0.12 mM of dTTP, 0.12 mM Cy5 or Cy3-dUTP), and 150 units of Klenow (New England Biolab) were added atop the plug and the plug was incubated at 37° C. in the dark for 2 hr.

Labeled DNA was eluted from the agarose and co-hybridized to Agilent microarray slides. More specifically, the embedded labeled DNA was electroeluted from the agarose plug in Spectra/Por dialysis tubing with a 12,000-14,000 MWCO (Spectrum) in 0.5×TBE at 110 volts at room temperature in the dark for 3 hr. The eluted DNA was then sonicated using a BioRuptor® sonicator (Diagenode, Denville, N.J.) to reduce the average size to 500 bp, and purified using the Qiagen PCR Cleanup Kit (Qiagen, Valencia, Calif.). The resulting DNAs from a control and an experimental sample that were differentially labeled with Cy-dUTP were mixed together and readied for microarray analysis.

The relative amount of ssDNA in the “S phase” sample was calculated as the ratio of fluorescent signals from the “S phase” sample to that from the “G1 phase” sample for each chromosome coordinate. As shown in FIG. 1B, ssDNA was detected near origins of replication in the HU 1 hr sample as observed previously. Interestingly, the ssDNA profile of the R 1 hr sample was virtually identical to that of the HU 1 hr sample, indicating that the single-stranded gaps remained unfilled behind the replication forks even after HU removal.

The DSBs were also labeled directly, in-gel. Sample preparation prior to labeling was performed as described above for the in-gel ssDNA labeling. DNA derived from an equal number of cells from the experimental sample (e.g., R 1 hr sample that contains DSBs) and from a control sample (non-replicating DNA without apparent breaks) was labeled differentially with Cy3- and Cy5-conjugated dUTPs. Cy-conjugated dUTP was incorporated at DNA ends in-gel with the End-It™ DNA End-Repair Kit (Epicentre Biotechnologies, Madison, Wis.), which utilizes both the polymerase and the 3′-5′ exonuclease activities of the T4 DNA polymerase. Thus, this in-gel “end-repair” reaction is able to process DNA ends with either 3′ or 5′ overhangs and generate (primarily) labeled blunt ends. (The End-It™ DNA End-Repair Kit also contains polynucleotide kinase, which adds a 5′-phosphate on the DNA end to facilitate cloning. This feature was not required for this example, but produced no ill effect.) For each sliver of agarose plug containing 108 cells in approximately 50 μl, 50 μl of End-Repair labeling mix (1× End-Repair buffer, 1 mM ATP, 0.5 mM dNTP mix, 1 μl of End-Repair enzyme mix) was added, and the plug was incubated at room temperature in the dark for 1 hr. The DNA was then eluted from agarose plugs and, after sonicating the DNA to reduce the average fragment size, co-hybridized to Agilent DNA microarrays. The relative levels of DSBs were expressed as the ratio of fluorescence signals from the experimental sample over those from the control sample. Data smoothing and peak identification were performed as described previously. Feng et al., 2009.

For microarray analysis, the experimental and control DNA samples were mixed for co-hybridization to the Agilent 4×4K Yeast ChIP DNA microarrays according to the manufacturer's recommendations. Data extraction was performed using Agilent's Feature Extraction™ software (Agilent, Santa Clara, Calif.). After removing those array spots flagged by the software as anomalous, the ratio of background-subtracted fluorescent signals from the experimental to the control sample was calculated for each probe. The resulting ratios for all the probe locations on each chromosome were smoothed with a 6 kb window using a Lowess smoothing algorithm as previously described. The relative levels of DSBs were expressed as the ratio of fluorescence signals from the experimental sample over those from the control sample. The “chromosome breakage profile” was generated by plotting the smoothed ratios against chromosome coordinates. Identification of peaks (local maxima) in the profiles was performed as previously described. Feng et al., 2009.

As a positive control, restriction digestion of DNA in agarose plugs was performed using BamHI, as described previously. Feng et al., 2009. Specifically, DNA embedded in agarose plugs was prepared as described herein from exponentially growing mec1 cells, and the DNA digested in-gel with the restriction enzyme BamHI. The digested DNA was then labeled in-gel with the End-It™ kit in the presence of Cy3-dUTP, and the undigested control DNA was labeled in the same fashion in the presence of Cy5-dUTP. The agarose plugs were pre-equilibrated in 10 mM Tris-HCl, pH 8.0, 0.1 mM EDTA, they were equilibrated in 1× End-Repair buffer (Epicentre, 33 mM Tris-acetate, pH 7.8, 66 mM KAc, 10 mM MgAc2, 5 mM dithiothreitol) at 5 ml/plug for 30 min at room temperature. Electroelution and subsequent analyses were performed identically as described above. Microarray analysis revealed the “chromosome breakage profile” of the cells containing BamHI ends (FIG. 2B). The results demonstrated that the mapped DSBs correlated nearly perfectly with the known BamHI sites in the genome. These results also demonstrated that the methods of in-gel preparation and labeling of DNA disclosed herein did not result in extensive site-specific shearing of the DNA: significant breakage elsewhere on the chromosome was not detected (FIG. 2B).

To examine the correlation between chromosome breakage and with replication fork progression, replication forks were allowed to travel different distances from the origins before HU treatment to generate collapsed forks at varying distances from the origins. After synchronizing mec1-1 cells at the G1/S boundary with a factor, the cell culture was split into five aliquots. The cells were released into S phase by pronase addition, and 200 mM HU added at 0, 5, 10, 15 or 20 minutes after the release to the five aliquots denoted as T0, T5, T10, T15, and T20, respectively. Each cell culture was exposed to HU for 1 hr before collecting the samples. The cells were then washed and transferred into fresh media without HU to let them “recover” for 1 hr. For clarity, samples are referred to by their aliquot number followed by the description of the treatment they received: “T#” refers to the elapsed time (in minutes) after a factor removal and before HU addition; “HU” for a 1 hr exposure to HU; and “R” for a 1 hr recovery from HU. Thus, T5-HU designates the sample in which HU was added at 5 min after the G1/S transition and which had been exposed to HU for 1 hr. Likewise, T20-R designates the sample in which HU was added at 20 min after G1/S transition, incubated for 1 hr in HU and then allowed to recover for 1 hr after HU was removed. The sites of chromosome breakage in samples recovering from HU were then measured (FIG. 4A). HU was added at different times after S phase began, and then the distribution of both ssDNA and double stranded breaks after cells recovered from HU were examined. Because it is the ssDNA at forks that contribute to chromosome fragility, the break sites are then found at a greater distance from these origins, as illustrated in FIG. 4B.

To further characterize the correlation between chromosome breakage and replication fork progression in the yeast model, DNA labeled in-matrix was prepared from each sample and subjected to Contour-clamped Homogeneous Electric Field (CHEF) gel eletrophoresis (FIG. 4C). CHEF gel analysis was performed as described previously. van Brabant et al., 7 Mol. Cell. 705 (2001); Feng et al., 2009. Electrophoresis was conducted at 14° C. for 25 hr with a switch time ramped from 60 sec to 120 sec at 200 volts. Standard procedures were employed to ethidium bromide stain and photograph the gel. In this gel system, as cells enter S phase their branched chromosomes become less visible in the gel, presumably because they are trapped in the well (most obviously seen at 20 minutes for the larger chromosomes; T20, lane 5). This property is more striking for the HU-treated samples (lanes 6-10) where replication forks are slowed. The reappearance of the large chromosomes in the T20-HU sample (lane 10) is consistent with many of the cells being able to complete replication when HU is added at this late time in S phase. Regardless of when in S phase the cells encountered HU, however, they all exhibited chromosome breakage after HU was removed from the culture (lanes 11-15). DSBs in the above mentioned samples were mapped, as a test of a correlation between fork progression and chromosome breakage: if forks had proceeded away from the origins during this experiment, the sites of ssDNA should reflect this fork migration; and, more importantly, the breakage sites to be at greater distances from the origins the later in S phase HU was added.

More specifically, chromosome breakage was mapped in the T20-R sample because it was the sample to which HU was added the latest in S phase (20 minutes after G1/S transition), and thus the sample in which replication forks had migrated the furthest. Chromosome breakage was also mapped in the control sample T0-R, where HU was added at the G1/S transition. Because chromosome breakage correlated with replication fork progression, the locations of DSBs in the T20-R sample should be different from those in the T0-R sample. As shown in FIG. 4D, the breakage profiles of the two samples were indeed different. There were two key observations: First, chromosome breakage at early replicating regions (near the unchecked origins) was reduced in cells exposed to HU later in S phase (FIG. 4D, coordinates 400-500 kb). Second, the peak positions of chromosome breakage were more broadly distributed in the regions replicated by forks from unchecked origins.

To quantify these observations regarding the change in breakage patterns in the yeast model, those chromosome positions where significant (above median level) breakage occurred were identified. Whether the break sites correlated with origin locations within a 6 kb distance was tested in the random simulation test. Neither the T0-R nor the T20-R sample showed correlation between the breakage sites and the unchecked origins, as shown in Table 1:

TABLE 1
Correlation between origins of replication and chromosome
breakage sites

| | T0-R break sites (639) | | T20-R break sites (788) | |
|---|---|---|---|---|
| | No. break | | No. break | |
| | sites near | P value of | sites near | P value of |
| Chromosomal | chromosomal | simulation | chromosomal | simulation |
| Feature | feature | test | feature | test |
| Unchecked | 25 | 1 | 39 | 1 |
| origins | | | | |
| (105) | | | | |
| Checked | 228 | 0.0032 | 231 | 0.8276 |
| origins | | | | |
| (210) | | | | |

Results of the random simulation tests for the correlation between chromosome breakage sites and various chromosome features within a 6 kb distance.
^{a}The numbers of break sites and other chromosome features are shown in brackets. The numbers of break sites found near most chromosomal features in the T20-Rsample are greater than those in the T0-R sample because the total number of significant (above median level) break sites in the T20-R sample is greater than in the T0-R sample (788 vs. 639).

The break sites in the T0-R sample were correlated with checked origins (p=0.0032); however, the break sites in the T20-R sample were not (p=0.8276) (Table 1). These observations demonstrate that chromosome breakage is less well correlated with origin locations when replication forks have had sufficient nucleotides and time to move into flanking regions, providing further support to the hypothesis that chromosome breakage is correlated with replication fork progression.

Whether the break sites are correlated with other chromosome features such as tRNAs, which have been shown to cluster with origins of replication were explored. See DiRienzi et al., 1 Genome Biol. Evol. 350 (2009). If break sites were indeed correlated with replication forks, the break sites should be correlated with tRNAs in the T0-R sample but not in the T20-R sample. The simulation test results confirmed this prediction: break sites are correlated with tRNAs in the T0-R sample (p=0.0012) but not with those in the T20-R sample (p=0.6135) (Table 1). Replication fork progression was also monitored by ssDNA mapping in the T0-HU and T20-HU samples. The results showed, once again, that the ssDNA profiles of the samples in HU prior to the occurrence of breakage were very similar to the breakage profiles (FIG. 4D). Thus, the present analyses conclusively demonstrated that: (a) chromosome breakage is correlated with replication fork progression; (b) ssDNA formation is detected prior to chromosome breakage taking place; and (c) sites of ssDNA formation predict the sites of chromosome breakage.

Random simulation test: To test for an association between break sites and chromosomal features within 6 kb, the distance between each break site and its nearest chromosomal feature of interest was first established. Distances between breaks and chromosomal features were measured from midpoint to midpoint. Breaks with chromosomal features less than 6 kb away were counted. The null distribution of the number of breaks with a given chromosomal feature within 6 kb was determined by randomizing the location of break sites and determining the distance between these random breaks and the chromosomal features. Break sites were randomized by randomly selecting an equal number of positions on the microarray. 10,000 simulations were run and in each run the number of random breaks with a given chromosomal feature within 6 kb were recorded. P values were obtained from the upper tail of the null distribution in which the number of breaks with a chromosomal feature within 6 kb was greater than or equal to that seen for the actual set of breaks. Chromosomal features used in these analyses were obtained from checked and unchecked origins of replication (see, e.g., Feng et al., 2006) and tRNAs (DiRenzi et al., 2009). A significantly greater percentage of breakage sites was found near the checked origins (41.6%) than near the unchecked origins (11.7%) (p<10-15) in a 2-sample test for equality of proportions with continuity correction. To test the correlation between breakage sites and checked origins more rigorously, a random simulation test was also performed, revealing that the breakage sites correlated with the checked origins (p<0.0001) but not with the unchecked origins (p=0.1851) within a 6 kb distance.

### Example 2

### In-Matrix Labeling to Detect ssDNA and DSBs in Mammalian Cell Chromosomes

To induce chromosome fragile sites in mammalian cell culture, HEK293T cells and GM066990 lymphoblasts were grown at a concentration of 5×106 cells/ml. An aliquot of 1 ml of cells was removed to serve as control cells in FACS analysis. The remaining cells are aliquoted and aphidicolin added to a final concentration of 0.2 μM to 0.6 μM. An equal volume of DMSO, the aphidicolin solvent, was added to mock control samples. Aphidicolin is a tetracyclic diterpene antibiotic with antiviral and antimitotical properties. Aphidicolin is a reversible inhibitor of eukaryotic nuclear DNA replication, blocking the cell cycle at early S phase. It is a specific inhibitor of DNA polymerase α, δ and ε in eukaryotic cells and in some viruses and an apoptosis inducer in HeLa cells.

After 24 hr incubation, a 1 ml aliquot of cells was removed for FACs analysis, and the remaining cells were harvested by centrifugation at 800 rpm for 8 min at room temperature (RT). After the aphidicolin step, cell concentration was adjusted to 10⁶ cells/ml. To generate two plugs of 10⁶ cells/plug, each sample requires at least 5 ml of cell culture. Each sample of 5 ml cell culture was washed by suspending in 2 ml ice-cold PBS buffer and pelleting at 800 rpm for 8 min at RT. Cells were resuspended in 500 μl ice-cold PBS and the cell suspension transferred to an eppendorf tube, and spun down at 2,000× rpm in table-top microfuge at 4° C. Cells were resuspended in 100 μl ice-cold PBS to a final concentration of ˜3×10⁷ cells/ml. A preparation of 1% low-melting agarose (Incert, Lonza Cat. #50123) was prepared in PBS, and equilibrated to 43° C. in a water bath. The cells were equilibrated at 43° C. for 5 min. An equal volume of 1% agarose was added to the cells, mixed, and cast in a mold. The agarose-embedded cells were than cooled on ice for 10 min to 15 min. Plugs were extruded into 6 ml lysis buffer/well (0.5 M EDTA, 1% Sarkosyl (N-lauryl sarkosin, Na salt), 200 μg/ml Proteinase K) in a six-well petri dish, and incubated at 50° C. over-night. The lysis buffer was aspirated and replaced with 6 ml fresh lysis buffer, and incubated again at 50° C. over-night. The plugs were rinsed in 6 ml TE pH 8.0 and incubated at RT for 1 hr, and this step repeated twice. The plugs can be stored long-term in fresh TE pH 8.0, but are otherwise suited for in-matrix labeling for ssDNA or DSBs.

In-gel labeling of ssDNA by Klenow fragment was achieved as follows. Agarose plugs were removed to fresh six-well petri plates, one plug/well, and washed twice with 5 ml TE 0.1 buffer (10 mM Tris-Hcl pH8, 0.1 mM EDTA) per well by incubating on a platform shaker for 15 min at RT, then washed twice with 5 ml reaction buffer (50 mM Tris-HCl pH 6.8, 5 mM MgCl₂, 10 mM β-mercaptoethanol) per well by incubating on a platform shaker for 30 min at RT. During the last washing step, a labeling reaction mix was prepared (12 μl Biotin-dNTPs (1 mM each), 20 μl 2.5× reaction buffer, 3 μl Klenow (50 KU/ml), 15 μl H₂O), based on an estimated gel volume of ˜50 μl. A small humidity chamber was prepared using a 1 ml blue tip box by placing about 50 ml water in the base of the box, then a piece of parafilm was placed on the platform inside (the plastic insert with 96 holes for tips), and the positions/sample names marked on the edges of the parafilm. Using a clean spatula, the plugs were placed on the parafilm according to the markings. Then 50 μl reaction mix was pipeted slowly onto each agarose plug. The reaction was then incubated for 2 hr to 3 hr, in the dark, at 37° C.

In-gel labeling of DSBs by end-repair enzyme mix was achieved as follows. Agarose plugs were moved to new six-well petri dishes, one plug/well, washed twice with 5 ml TE 0.1 per well by incubating for 15 min on a platform shaker at RT, then washed twice with 5 ml reaction buffer (33 mM Tris-acetate pH 7.8, 6 mM potassium acetate, 10 mM magnesium acetate, 0.5 mM DTT) by incubating on a platform shaker for 30 min at RT. During the last wash step, a labeling reaction mix was prepared (5 μl End-repair buffer (10×), 12 μl Biotin-dNTPs (1 mM each), 10 mM ATP, 3 μl End-It enzyme mix, 20 μl H2O), based on an estimated gel volume of ˜50 μl. A small humidity chamber was prepared as described above. Using a clean spatula, the plugs were placed on the parafilm according to the markings. Then 50 μl reaction mix was pipeted slowly onto each agarose plug. The reaction was then incubated for 1.5 hr, in the dark, at RT.

After in-gel labeling, agarose was digested by agarase digestion as follows. Each agarose plug was transferred to a pre-weighed empty eppendorf tube and re-weighed to obtain the weight of the agars plug. The agarose plugs were then melted by incubating for 10 min in a 70° C. water-bath. The tubes were then microfuged at maximum speed for 1 min and placed in a 47° C. heat-block for 1 min. An aliquot of 0.29 U (1 μl) AgarAce (Promega) per 50 mg molten agarose was added to each tube, and the tubes incubated for 1 hr at 47° C. The tubes were then cooled on ice for 5 min and microfuged at maximum speed for 20 min at 4° C. The supernatants were then transferred to new eppendorf tubes.

The DNAs were then sonicated and processed as follows. The volume in each tube was estimated, and if the contents exceeded 300 μl the contents distributed evenly into eppendorf tubes such that the maximum volume did not exceed 300 μl. Each sample was sonicated to shear the DNA fragments using a Bioruptor using 20 cycles, 30 sec on, 30 sec off, on the “high” setting. DNA should be ˜≦500 bp at this point. A Qiagen PCR cleanup kit was used to clean the samples, following the instructions. Each sample was eluted with 32 μl of elution buffer (EB) (10 mM Tris-HCl pH 8.5). An aliquot of 2 μl was removed for analytical agarose gel electrophoresis.

Streptavidin beads were used to purify labeled DNA by adding 170 μl TE to each sample to a final volume of 200 μl and mixing with the beads. To prepare Dynabeads, 600 μM270 Dynabeads (Invitrogen, Carlsbad, Calif.) suspension was placed on a magnet for 2 min, and the supernatant removed. 200 μl of 2×B&W buffer (10 mM Tris-HCl pH 7.5, 1 mM EDTA, 2M NaCl) was added to each tube, mixed by pipetting up-and-down, and the tubes magneted. This process was repeated four to six times, then 200 μl 2×B&W buffer mixed in each tube, which was then aliquoted into two portions, magneted, and the supernatants removed. After Dynabeads were prepared, 200 μl 2×B&W buffer and 200 μl DNA added to each tube, which were then incubated on a rotator for 15 min at RT. Each reaction was then washed thrice with 400 μl 1×B&W buffer, and thrice with 200 μl EB.

End repair was conducted on the bead-bound DNA using End-It™ repair kit, Epicentre® (Illumina) by adding, to each tube, 5 μl 10× end repair buffer, 5 μl dNTP mix (2.5 mM each), 5 μl 10 mM ATP, 34 μl H2O, 1 μl End it enzymes (total volume 50 μl/tube), mixing well and incubating for 45 min at RT. Each sample was then washed 4 times with 200 μl 1×R&W, twice with 200 μl EB, after which the beads were resuspended in 15 μl EB.

For A-tailing, 15 μl of each end-repaired DNA was mixed with 5 μl 10×PCR buffer with Mg2+ (Roche catalog #12779800), 1.5 μl 50 mM MgCl₂, 0.25 μl 100 mM dATP (Invitogen P/N55082), 0.25 μl Taq (NEB M0267S, 5 U/μl), 28 μl H2O (total 50 μl), and incubated in the PCR machine for 30 min at 70° C. This treatment was followed by washing four times with 200 μl 1×B&W and twice with 200 μl EB. The bead-bound DNA was the resuspended in 5 μl EB.

Adaptors were prepared according to Illumina's recommendation. Briefly, all four promers were prepared (Solexa-1_top, 1_bottom, PlexP2_top and PlexP2_bottom) at 100_{—}1M in TE pH 8.0 and stored at 4° C.; then 50 μl each Solexa-1_top and Solexa-1_bottom mixed, and 50 μl each Solexa_PlexP2_top and Solex_PlexP2_bottom mixed to produce adaptors Solex-1 and Solexa-2, respectively; the tubes incubated in the PCR machine for 5 min at 95° C. After incubation, the PCR machine was turned off and left to cool gradually to RT (about 1.5 hr) and the Solexa adaptors stored at −20° C. Ligation was achieved by mixing 5 μl end-repaired, A-tailed DNA with 1 μl 50 μM Solexa-1, 1 μl 50 μM Solexa-2, 8 μl 2× Quick buffer, 1 μl Quick T4 DNA ligase (NEB M2200S) (total 16 μl), and incubating for 30 min at RT. The samples were then washed 4 times with 200 μl 1×R&W, twice with 200 μl EB, after which the beads were resuspended in 50 μl PCR mix.

PCR enrichment of the bead-bound DNA was prepared by adding 5 μl 10×Pfu buffer, 5 μl dNTPs (2.5 mM each), 1.25 μl 10 μM Solexa-F, 1.25 μl 10 μM Solexa-R, 0.5 μl Pfu (New England Biolabs), 37 μl H2O, and the reaction carried out at 98° C. for 1 min, followed by eighteen cycles of 98° C., 10 sec; 65° C., 30 sec; 72° C., 2 min; and the samples stored at 4° C. These samples were suited for sequencing and/or microarray analysis.

In similar studies using hydroxyurea-treated human cell lines, GM06990 lymphoblasts and GM693-cc1 fibroblasts and the ssDNA labeling procedures described herein, origins of replication were identified. These sites, at which DNA replication initiates, have been linked to chromosome fragility. Di Rienzi et al., 1 Genome Biol. Evol. 350 (2009). Known origins were enriched for ssDNA and identified, for example, the Epstein Barr Virus (used to transform the lymphoblasts) origin and the D-loop region in the mitochondrial genome were identified. Additionally, 2,000 putative origins were identified in the nuclear genome, and may be confirmed using independent methods.

We claim:

1. A method for genome-wide detection of chromosome fragile sites characterized by regions of single-stranded DNA (ssDNA) or double stranded breaks (DSB) in eukaryotic cellular chromosomal DNA, the method comprising:
(a) obtaining a matrix in which a plurality of cells have been embedded and said cells' chromosomes have been made accessible to labeling reagents;
(b) within the matrix, contacting the chromosomal DNA of the cells with labeling reagents to label directly the DNA at ssDNA regions or at sites of DSB;
(c) isolating the labeled DNA from the matrix; and
(d) detecting the labeled DNA, wherein the detection of labeled DNA permits identification of the location of the ssDNA region or DSB sites and/or the magnitude of labeling at such sites.

2. The method of claim 1, further comprising the step, after labeling step (b), of fragmenting the chromosomal DNA.

3. The method of claim 2, wherein the fragmenting step is performed before or after isolating step (c)

4. The method of claim 2, wherein the fragmenting step comprises mechanical shearing, enzymatic cleavage, or chemical cleavage.

5. The method of claim 4, wherein the fragmenting step produces fragments having an average size of about 400 bp to about 600 bp, inclusive

6. The method of claim 1, wherein the step of isolating labeled DNA from the matrix comprises electroelution or digestion of the matrix.

7. The method of claim 1, wherein detecting step (d) comprises contacting the labeled DNA with a microarray, or analyzing labeled DNA by high throughput DNA sequencing.

8. The method of claim 7, wherein the detecting step permits detection of both the location of the chromosome fragile site and the magnitude of labeling at the chromosome fragile site.

9. The method of claim 1 wherein the labeling reagents comprise random primers that permit labeling at ssDNA chromosomal regions.

10. The method of claim 1 wherein the labeling reagents comprise Klenow fragment or T4 polymerase

11. The method of claim 1 wherein the labeling reagents comprise at least one fluorescently labeled nucleotide.

12. The method of claim 11, wherein the fluorescently labeled nucleotide comprises fluorescently labeled dUTP.

13. The method of claim 1, wherein the cell is a yeast cell or a mammalian cell.

14. The method of claim 13, wherein the mammalian cell is a human cell.

15. The method of claim 13, wherein the cell is a cell of a mammalian cell line.

16. The method of claim 13, wherein the cell is obtained from a mammal suffering from or suspected of suffering from a chromosomal breakage disorder.

17. The method of claim 1, wherein the matrix is agarose.

18. A method for mapping chromosome fragile sites in a eukaryotic cell, the method comprising:
(a) contacting a plurality of eukaryotic cells with an inhibitor of DNA replication;
(b) embedding the cells in a matrix;
(c) within the matrix, contacting chromosomal DNA of the cells with labeling reagents for a time and under conditions sufficient to permit direct labeling of the DNA at single-stranded regions and/or at sites of double-stranded breaks;
(d) isolating labeled DNA from the matrix;
(e) detecting labeled DNA, wherein the detection of labeled DNA indicates the location of single-stranded regions and/or sites of double-stranded breaks in the chromosomal DNA of said cells, whereby one or more chromosome fragile sites are mapped.

19. The method of claim 17, further comprising the step of removing the inhibitor of DNA replication before embedding the cells in a matrix.

20. The method of claim 18, wherein the labeling reagents comprise random primers and biotinylated dUTP.

21. The method of claim 18, further comprising, after labeling step (c), fragmenting the chromosomal DNA.

22. The method of claim 21, further comprising the step, after the fragmenting step, of contacting the fragments with streptavidin bound to a solid support, whereby labeled DNA fragments are isolated.

23. The method of claim 18, wherein the detecting step (e) comprises contacting isolated, labeled DNA with a microarray, or subjecting isolated, labeled DNA to a high throughput DNA sequencing regimen.

24. A kit for mapping chromosome fragile sites in the chromosomal DNA of a eukaryotic cell comprising:
a matrix suitable for embedding eukaryotic cells; and
reagents for direct labeling of the DNA at single-stranded regions and/or at sites of double-stranded breaks.

25. The kit of claim 24, further comprising control cells and/or control DNA.

26. The kit of claim 24, further comprising at least one microarray.
